(19) United States
(12) Patent Application Publication
(10) Pub. No.: US 2013/0123255 A1
(43) Pub. Date: May 16, 2013

(54) COMBINATION OF A PI3K INHIBITOR AND A MEK INHIBITOR

(71) Applicant: CHUGAI SEIYAKU KABUSHIKI KAISHA, Tokyo (JP)

(72) Inventors: Hiromi Tanimura, Tokyo (JP); Yasushi Tomii, Tokyo (JP); Kiyoaki Sakata, Tokyo (JP); Miyuki Yoshida, Tokyo (JP); Hiroshi Tanaka, Tokyo (JP); Nobuya Ishii, Tokyo (JP); Osamu Kondoh, Tokyo (JP)

(73) Assignee: CHUGAI SEIYAKU KABUSHIKI KAISHA, Tokyo (JP)

(21) Appl. No.: 13/669,782

(22) Filed: Nov. 6, 2012

Related U.S. Application Data
(60) Provisional application No. 61/558,034, filed on Nov. 10, 2011.

(51) Int. Cl. A61K 31/5377; A61P 35/00

(52) U.S. Cl. 514/234.2

(57) ABSTRACT

The invention relates to a method for the treatment of a patient with a proliferative disease including solid tumors, hematological malignancies and hyperplasia comprising administering a therapeutic combination to said patient wherein the therapeutic combination comprises a therapeutically effective amount of a compound of formula (I) or a pharmaceutically acceptable salt thereof, and a therapeutically effective amount of a compound of formula (II) or a pharmaceutically acceptable salt thereof.

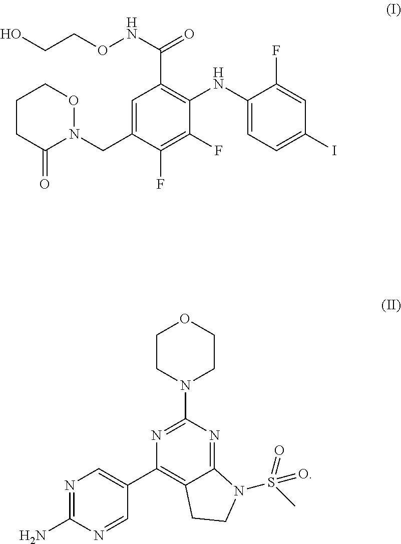 [SMILES: O=C(NOCCO)c1cc(CN2OCCCC2=O)c(F)c(F)c1Nc1ccc(I)cc1F ; CS(=O)(=O)N1CCc2c(-c3cnc(N)nc3)nc(N3CCOCC3)nc21]

### CROSS-REFERENCE TO RELATED APPLICATIONS

This application claims priority from U.S. Provisional Application No. 61/558,034, filed Nov. 10, 2011. The entire contents of the aforementioned application is incorporated herein by reference.

### FIELD OF THE INVENTION

The invention relates to methods of treating a patient with a proliferative disease including solid tumors, hematological malignancies and hyperplasia with a combination of an inhibitor of mitogen activated protein kinase (MEK) and an inhibitor of phosphatidylinositol 3-kinase (PI 3-kinase or PI3K) described herein.

### BACKGROUND OF THE INVENTION

As a signal transduction pathways which regulate the process of signal transduction from the cell surface to the nucleus, the Ras signaling pathway and the PI3K (Phosphatidylinositol 3 kinase) pathway have been known. The PI3K pathway is either activated via cell surface receptors or indirectly by Ras.

The MAPK (mitogen-activated protein kinase) cascade which comprises three kinases, namely, Raf, MEK (MAPK or ERK kinase), and ERK (extracellular stimulus regulated kinase), is a key component of the Ras signaling pathway. The cascade starts with the activation of Ras and in response to extracellular signals, plays an important role in regulating cell proliferation, differentiation, and transformation (Person G., et al., Endocrine Rev., 22, 153-183 (2001); Bryan A. Ballif et al., Cell Growth & Differentiation, 12, 397-408 (2001); Cobb M H., Prog. Biophys. Mol. Biol., 71 479-500 (1999); Lewis T S., et al., Adv. Cancer Res., 74 49-139 (1998); Kolch W, Biochem. J., 351,289-305 (2000); Judith S Sebolt-Leopold, Oncogene, 19, 6594-6599 (2000); Roman Herrera et al., Treds in Molecular Medicine, 8, S27-S31 (2002)).

One of the above three kinases, MEK is a dual-specificity kinase. Activated MEK phosphorylates ERK1 and ERK2 on tyrosine (185) and threonine (183) residues (Anderson N G et al., Nature, 343, 651-653 (1990); Seger R et al., FASEG J, 9 716-735 (1995)). The MEK-mediated ERK phosphorylation results in not only ERK activation but also translocation of ERK to the nucleus. Activated ERK (MAPK) activates various substrates, for example, transcription factors in the cytoplasm and nucleus, and as a result, the activation leads to cellular changes (proliferation, differentiation, and transformation) depending on the extracellular signal.

Constitutive activation of the MEK/MAPK pathway is shown to be associated with the neoplastic phenotypes of a relatively large number of cancer cell types (Hoshino R. et al., Oncogene, 18, 813 (1999); Kim S C., et al., Blood, 93, 3893 (1999); Morgan M A. Et al., Blood, 97, 1823 (2001)).

In addition, constitutive activation of MEK has been reported to result in cellular alteration (transformation or canceration) (Cowley S. et al., Cell, 77, 841-852 (1994); Mansour S J. et al., Science, 265, 966-970 (1994)).

Furthermore, studies of MEK inhibitors (PD98059 and others) have revealed that MEK inhibition not only results in impaired cell proliferation, but also has impact on various cellular events, including cell differentiation, apoptosis, and angiogenesis (Dudley D T. et al., Proc. Natl. Acad. Sci. USA, 92, 7686-7689 (1995); Alessi D R. et al., J. Biol. Chem., 270, 27489-27494 (1995); Pages G. et al., J, Proc. Natl. Acad. Sci. USA., 90, 8319-8323 (1993); Pang L. et al., J. Biol. Chem., 270, 13585-13588 (1995); Finalay D. et al., Cell Death Differ. 7, 303-313 (2000); Holmstrom T H et al., Mol. Cell. Biol., 19, 5991-6002 (1999); Elliceiri B P et al., J. Cell Biol., 141, 1255-1263 (1998); Milanini J et al., J. Biol. Chem., 273, 18165-18172 (1998)).

From the above, MEK, one of the major mediators in the MAPK cascade, can serves as a potential target for therapeutic agents used in treating diseases caused by aberrant cell proliferation.

Until now, various MEK inhibitors have been reported, and as one of MEK inhibitors, N-alkoxy-2-phenylamino-benzamide derivatives which have an alkoxy residue as the substituent on the amide nitrogen atom and have various substituent groups at 5^{th} position of the benzamide ring have been reported (WO 1998/37881, WO 1999/01426, WO 2000/42003, WO 2001/68619, WO 2002/06213, WO 2006/11466). As one of such N-alkoxy-2-phenylamino-benzamide derivatives, 3,4-difluoro-2-(2-fluoro-4-iodo-phenylamino)-N-(2-hydroxy-ethoxy)-5-(3-oxo-[1,2]oxazinan-2-ylmethyl)-benzamide which is represented by the formula (I):

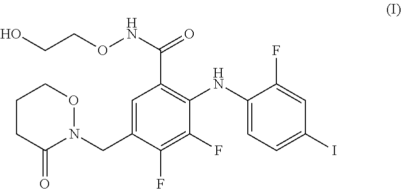 [SMILES: O=C(NOCCO)c1cc(CN2OCCCC2=O)c(F)c(F)c1Nc1ccc(I)cc1F]

was disclosed in WO 2006/11466. This compound has been reported to have an unexpectedly strong MEK inhibitory effect as well as effects of suppressing tumor growth and of suppressing the onset of arthritis, and then serve as preventive or therapeutic agents for cancers and arthritis with improved biological utility.

On the other hand, phosphatidylinositol 3-kinase (PI3K) is known to be activated by stimulation by growth factors, hormones and the like, to activate Akt and PDK1, and to be involved in survival signals that inhibit cell death, cytoskeleton, glucose metabolism, vesicular transport and the like. In addition, the phosphatidylinositols phosphorylated at position 3 that are formed by PI3K function serves as messengers of these information transfer systems (Eur. J. Biochem. 268, 487-498 (2001); Biochem. J. 333, 471-490 (1998); J. C. B., 155(1), 19-25 (2001) and the like).

PI3K is categorized into three classes consisting of Class I, Class II and Class III according to the type of phosphatidylinositols serving as a substrate.

Although Class I enzymes form phosphatidylinositol (3,4,5)-triphosphate [PI(3,4,5)P3] by using phosphatidylinositol (4,5)-bisphosphate [PI(4,5)P2] as a substrate in vivo, it can use phosphatidylinositol (PI) and phosphatidylinositol (4)-phosphate [PI(4)P] as substrates in vitro. Further, Class I enzymes are categorized into Class Ia and Ib according to the activation mechanism. Class Ia includes the p110α, p110β and p110δ subtypes, and each forms a heterodimer complex with a p85 regulatory subunit and is activated by a tyrosine kinase receptor and the like. Class 1b includes a p110γ subtype that is activated by the βγ subunit (Gβγ) of a trimer G protein, and forms a heterodimer with a p101 regulatory subunit.

Recently, a gene amplification of PIK3CA encoding p110α, constitutive activation due to mutation, and high expression of p110α at the protein level have been reported in numerous types of cancers (and particularly ovarian cancer, colon cancer and breast cancer). As a result, inhibition of apoptosis by constitutive activation of survival signals is believed to be partially responsible for the mechanism of tumorigenesis (Nature Genet. 21, 99-102, (1999); Science, 304, 554, (2004); Cancer, 83, 41-47 (1998)).

In addition, the deletion or mutation of PTEN, a phospholipid phosphatase which hydrolyzes PI (3,4,5) P3 that is one of the products of PI3K, has been reported in numerous cancers. Since PTEN functions as a suppressor of PI3K as a result of using PI (3,4,5) P3 as a substrate, deletion or mutation of PTEN is thought to lead to activation of PI3K in the PI3K signal.

For these reasons, useful anticancer action is expected to be obtained by particularly inhibiting the activity of p110α in cancers with elevated PI3K activity. As a compound having PI3K inhibiting activity, Wortmannin (Yano H. et al., J. Biol. Chem., 268, 25846, 1993) and LY294002 (Vlahos C J et al., J. Biol. Chem., 269, 5241, 1994) have been known. Further, it has been reported in WO 2008/018426 that 2-morpholin-4-yl-6,7-dihydro-5H-pyrrolo[2,3-d]pyrimidine derivatives have a superior PI3K inhibitory effect allowing it to be a useful drug for the prevention or treatment of cancer.

As one of such a derivative, 5-(7-methanesulfonyl-2-morpholin-4-yl-6,7-dihydro-5H-pyrrolo[2,3-d]pyrimidin-4-yl)-pyrimidin-2-ylamine (WO 2008/018426), represented by the formula (II):

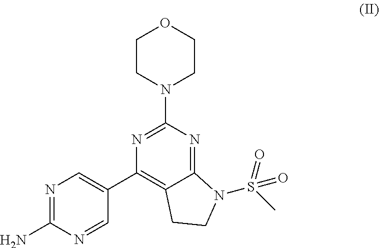 [SMILES: CS(=O)(=O)N1CCc2c(-c3cnc(N)nc3)nc(N3CCOCC3)nc21]

has a particularly superior PI3K inhibitory activity as well as superior stability in a body and water solubility, and useful anticancer activity, which is expected to be obtained by inhibiting the activity of p110α in particular in cancers with elevated PI3K activity.

Many cancers (e.g., melanoma, colorectal, pancreatic, ovarian, NSCLC, and thyroid cancers) have a high and overlapping frequency of oncogenic mutations that activate both Ras and PI3K pathways. Furthermore, in tumor cells, inhibition of one activated pathway can result in activation of the other pathway; therefore, inhibition of both Ras and PI3K pathways represents a new anti-cancer strategy. Thus, combined MEK and PI3K inhibition is an exciting approach to treat cancers.

This approach has a dual benefit: it has the potential to increase the initial tumor response rate in tumors driven by multiple oncogenic events, as well as to decrease the rates of acquired resistance that could occur with either agent alone. This is due to the inhibition of the activating compensatory pathways, which would then prolong the activity of the combination over the activity seen by either agent alone.

Such a combination of a MEK inhibitor and a PI3K inhibitor have been proposed in US 2011/0086837 and Meng J. et al., PLoS ONE, 5, 1-10 (2010).

### SUMMARY OF THE INVENTION

The invention relates to a method for the treatment of a patient with a proliferative disease including solid tumors, hematological malignancies and hyperplasia comprising administering a therapeutic combination to said patient wherein the therapeutic combination comprises a therapeutically effective amount of a compound of formula (I) or a pharmaceutically acceptable salt thereof, and a therapeutically effective amount of a compound of formula (II) or a pharmaceutically acceptable salt thereof.

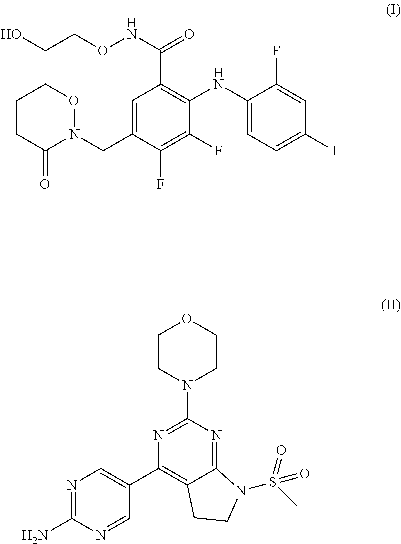 [SMILES: O=C(NOCCO)c1cc(CN2OCCCC2=O)c(F)c(F)c1Nc1ccc(I)cc1F ; CS(=O)(=O)N1CCc2c(-c3cnc(N)nc3)nc(N3CCOCC3)nc21]

The invention also relates to a pharmaceutical kit of parts comprising:

a) a pharmaceutical composition comprising a therapeutically effective amount of a compound of formula (I),

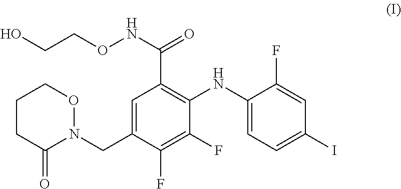 [SMILES: O=C(NOCCO)c1cc(CN2OCCCC2=O)c(F)c(F)c1Nc1ccc(I)cc1F]

or a pharmaceutically acceptable salt thereof,

b) a pharmaceutical composition comprising a therapeutically effective amount of a compound of formula (II),

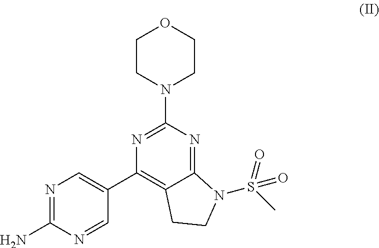 [SMILES: CS(=O)(=O)N1CCc2c(-c3cnc(N)nc3)nc(N3CCOCC3)nc21]

or a pharmaceutically acceptable salt thereof, and optionally

c) instructions for dosing regimen.

The present invention also relates to a method for the treatment of a patient with a proliferative disease including solid tumors, hematological malignancies and hyperplasia comprising the steps of providing the pharmaceutical kit of parts as above, and administering to said patient a pharmaceutical composition comprising a therapeutically effective amount of the compound of formula (I) or a pharmaceutically acceptable salt thereof and a pharmaceutical composition comprising a therapeutically effective amount of the compound of formula (II) or a pharmaceutically acceptable salt thereof.

With the method of the present invention, the synergistic effect on a proliferative disease including solid tumors, hematological malignancies and hyperplasia can be obtained as compared with when the compound of formula (I) or the compound of formula (II) is administered alone. Further, with the method of the present invention, an adverse effect such as decrease of body weight of the patient can be reduced.

### BRIEF DESCRIPTION OF THE DRAWINGS

FIG. 1A shows in vitro synergistic effect of compound of formula (I) and compound of formula (II).

FIG. 1B shows in vitro synergistic effect of compound of formula (I) and compound of formula (II).

FIG. 2 shows apoptosis induction by compound of formula (I) and compound of formula (II).

FIG. 3 shows PI3K and MAPK pathway inhibition by compound of formula (I) and compound of formula (II).

FIG. 4 shows effect of compound of formula (I) and compound of formula (II) on tumor growth in HCT-116 (PI3Kα^{H1047R}, KRAS^{G13D}) mutant xenograft model.

FIG. 5 shows effect of compound of formula (I) and compound of formula (II) on relative body weight in HCT-116 (PI3Kα^{H1047R}, KRAS^{G13D}) mutant xenograft model.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

The terms “comprise” and “comprising” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treat” and “treatment” can also mean prolonging survival as compared to expected survival if not receiving treatment. Those in need of treatment include those already with the condition or disorder as well as those prone to have the condition or disorder or those in which the condition or disorder is to be prevented.

The terms “proliferative disease” include solid tumors, hematological malignancies and hyperplasia.

The terms “solid tumors” include but not restricted to breast cancer, colon cancer, ovarian cancer, lung cancer, pancreatic cancer, liver cancer, uterine cancer, brain tumor, and prostatic cancer.

The terms “hematological malignancies” include but not restricted to chronic myeloid leukemia (CML), acute lymphocyte leukemia (ALL), and acute myeloid leukemia (AML).

The terms “pharmaceutically acceptable” indicate that the substance or composition must be compatible chemically and/or toxicologically, with the other ingredients comprising a formulation, and/or the mammal being treated therewith.

The terms “pharmaceutically acceptable salt” refer to pharmaceutically acceptable non-toxic organic or inorganic salts with the compounds of formula (I) or formula (II) of the invention. Exemplary salts include, but are not limited, hydrochloride, hydrobromate, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate, and 1,1′-methylene-bis-(2-hydroxy-3-naphthoic acid) salt with the compound of formula (I) or the compound of formula (II).

The pharmaceutically acceptable salt may be prepared by any suitable method available in the art, for example, by treatment of the compound of formula (I) or the compound of formula (II) with an organic or inorganic acid as exemplified above.

The invention relates to a method for the treatment of a patient with a proliferative disease including solid tumors, hematological malignancies and hyperplasia comprising administering a therapeutic combination to said patient wherein the therapeutic combination comprises a therapeutically effective amount of a compound of formula (I) or a pharmaceutically acceptable salt thereof, and a therapeutically effective amount of a compound of formula (II) or a pharmaceutically acceptable salt thereof.

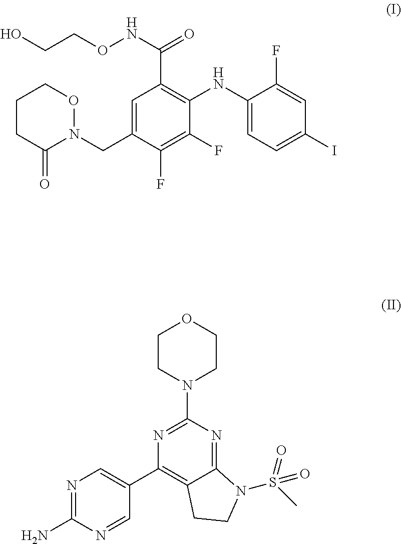 [SMILES: O=C(NOCCO)c1cc(CN2OCCCC2=O)c(F)c(F)c1Nc1ccc(I)cc1F ; CS(=O)(=O)N1CCc2c(-c3cnc(N)nc3)nc(N3CCOCC3)nc21]

The invention also relates to a pharmaceutical kit of parts comprising:

a) a pharmaceutical composition comprising a therapeutically effective amount of a compound of formula (I),

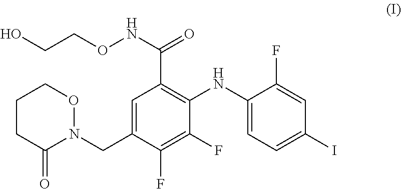 [SMILES: O=C(NOCCO)c1cc(CN2OCCCC2=O)c(F)c(F)c1Nc1ccc(I)cc1F]

or a pharmaceutically acceptable salt thereof,

b) a pharmaceutical composition comprising a therapeutically effective amount of a compound of formula (II),

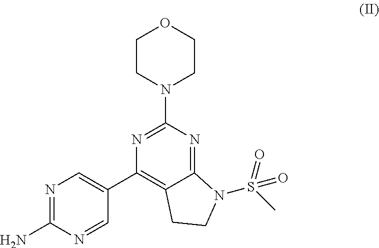 [SMILES: CS(=O)(=O)N1CCc2c(-c3cnc(N)nc3)nc(N3CCOCC3)nc21]

or a pharmaceutically acceptable salt thereof, and optionally

c) instructions for dosing regimen.

Further, the invention also relates to a method for the treatment of a patient with a proliferative disease including solid tumors, hematological malignancies and hyperplasia comprising the steps of providing the pharmaceutical kit of parts as above, and administering to said patient a pharmaceutical composition comprising a therapeutically effective amount of the compound of formula (I) or a pharmaceutically acceptable salt thereof and a pharmaceutical composition comprising a therapeutically effective amount of the compound of formula (II) or a pharmaceutically acceptable salt thereof.

The compound of formula (I) to be used in the method herein may be prepared following the methods described in WO 2006/011466 or US 2010-0197676 (Example 36), the content of which is incorporated herein by reference in its entirety. The compound of formula (II) to be used in the method herein may be prepared following the methods described in WO 2008/018426 or US 2010-0069629 (Example 1-D-02), the content of which is incorporated herein by reference in its entirety.

In the method of the present invention, the compound of formula (I) and the compound of formula (II) include all stereoisomers, geometric isomers, tautomers, metabolites and pharmaceutically acceptable salts thereof.

The compound of formula (I) and the compound of formula (II) may also exist in different tautomeric forms, and all such forms are embraced within the scope of the invention. The term “tautomer” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the method of the present invention, a metabolite of the compound of formula (I) or the compound of formula (II) can be also used. A “metabolite” is a product produced through metabolism in the body of a specified compound or salt thereof. Metabolites of a compound may be identified using routine techniques known in the art and their activities may be determined using tests known in the art. Such products may result for example from the oxidation, reduction, hydrolysis, amidation, deamidation, esterification, deesterification, enzymatic cleavage, and the like, of the administered compound. Accordingly, the method of the present invention includes metabolites of the compound of formula (I) or the compound of formula (II), including compounds produced by a process comprising contacting the compound of formula (I) or the compound of formula (II) with a mammal for a period of time sufficient to yield a metabolic product thereof.

The compound of formula (I) and the compound of formula (II) may exist in unsolvated or solvated form with pharmaceutically acceptable solvents. Examples of solvents that form the solvates include, but are not limited to, water; an alcohol such as isopropanol, ethanol, methanol; sulfoxide such as DMSO; an ester such as ethyl acetate; an acid such as acetic acid; an amine such as ethanolamine, etc. It is intended that the invention embraces both solvated and unsolvated forms.

Further, the compound of formula (I) and the compound of formula (II) may also exist in a hydrate. The term “hydrate” refers to the complex where the solvent molecule is water.

A typical, non-limiting, process for forming a solvate or a hydrate involves dissolving the compound of formula (I) or the compound of formula (II) in desired amounts of the desired solvent (pharmaceutically acceptable solvent or water or mixtures thereof) at higher than ambient temperature, and cooling the solution at a rate sufficient to form crystals, which are then isolated by standard methods. Analytical techniques such as, for example IR spectroscopy, show the presence of the solvent (or water) in the crystals as a solvate (or hydrate).

The compound of formula (I) and the compound of formula (II) may also be isotopically-labeled, i.e., one or more atoms may be replaced by an atom having an atomic mass or mass number different from the atomic mass or mass number usually found in nature. All isotopes of any particular atom as specified are contemplated within the scope of the compounds of the invention, and their uses. Exemplary isotopes that can be incorporated into the compound of formula (I) and the compound of formula (II) include isotopes of hydrogen, carbon, nitrogen, oxygen, sulfur, fluorine, and iodine, such as ²H, ³H, ¹¹C, ¹³C, ¹⁴C, ¹³N, ¹⁵N, ¹⁵O, ¹⁷O, ¹⁸O, ³⁵S, ¹⁸F, ¹²³I and ¹²⁵I. Certain isotopically-labeled compounds of formula (I) or formula (II) (e.g., those labeled with ³H and ¹⁴C) are useful in compound and/or substrate tissue distribution assays. Tritiated (³H) and carbon-14 (⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds can generally be prepared by substituting an isotopically labeled reagent for a non-isotopically labeled reagent.

In the method of the present invention, the compound of formula (I) or a pharmaceutically acceptable salt thereof, and the compound of formula (II) or a pharmaceutically acceptable salt thereof are administered as a therapeutic combination comprising a therapeutically effective amount of the compound of formula (I) or a pharmaceutically acceptable salt thereof, and a therapeutically effective amount of the compound of formula (II) or a pharmaceutically acceptable salt thereof, i.e., in the form of a pharmaceutical composition comprising a therapeutically effective amount of the compound of formula (I) or a pharmaceutically acceptable salt thereof in combination with a pharmaceutical composition comprising a therapeutically effective amount of the compound of formula (II) or a pharmaceutically acceptable salt thereof; or in the form of a pharmaceutical composition comprising a therapeutically effective amount of the compound of formula (I) or a pharmaceutically acceptable salt thereof and a therapeutically effective amount of the compound of formula (II) or a pharmaceutically acceptable salt thereof.

In the method of the present invention, the pharmaceutical composition comprising the compound of formula (I) or a pharmaceutically acceptable salt thereof; the pharmaceutical composition comprising the compound of formula (II) or a pharmaceutically acceptable salt thereof; and the pharmaceutical composition comprising the compound of formula (I) or a pharmaceutically acceptable salt thereof and the compound of formula (II) or a pharmaceutically acceptable salt thereof may be administered orally or parenterally (such as intravenously, intramuscularly, subcutaneously, rectally, nasally, intracisternally, vaginally, abdominally, intracystically or locally), but preferably orally administered.

In the method of the present invention, examples of the pharmaceutical composition for oral administration include tablets, capsules, granules, powders, pills, aqueous or non-aqueous oral solutions and suspensions. Examples of the pharmaceutical composition for parenteral administration include injections, ointments, gels, creams, suppositories, oral or nasal sprays, emulsions, oily agents and suspending agents, as well as parenteral solutions filled into containers suitable for administration in individual small doses. In addition, the administration form can be adapted to various administration methods including controlled-release formulations in the manner of subcutaneous transplants.

The pharmaceutical composition can be produced according to any of the methods well known in the art of pharmacy as in Remington's Pharmaceutical Sciences 18thEd. (1995) Mack Publishing Co., Easton, Pa., using additives ordinarily used in pharmaceutical preparations, examples of which include vehicles, lubricants (coating agents), binders, disintegrators, stabilizers, correctives, diluents, surfactants and emulsifiers.

Examples of vehicles include starches such as starch, potato starch and cornstarch, lactose, crystalline cellulose and calcium hydrogen phosphate.

Examples of coating agents include ethyl cellulose, hydroxypropyl cellulose, hydroxypropyl methyl cellulose, shellac, talc, Carnauba wax and paraffin.

Examples of binders include polyvinyl pyrrolidone, Macrogol and the same compounds as listed for the aforementioned vehicles.

Examples of disintegrators include the same compounds as those listed for the aforementioned vehicles and chemically-modified starches and celluloses such as cross carmellose sodium, sodium carboxymethyl starch or crosslinked polyvinyl pyrrolidone.

Examples of stabilizers include paraoxybenzoic acid esters such as methyl paraben or propyl paraben; alcohols such as chlorobutanol, benzyl alcohol or phenylethyl alcohol; benzalkonium chloride; phenols such as phenol or cresol; thimerosal; dehydroacetic acid; and sorbic acid.

Examples of correctives include ordinarily used sweeteners, sour flavorings and fragrances.

Examples of surfactants and emulsifiers include Polysorbate 80, Polyoxyl 40 Stearate and Lauromacrogol.

In addition, examples of solvents which can be used for producing liquid preparations include ethanol, phenol, chlorocresol, purified water and distilled water.

In the method of the present invention, a therapeutically effective amount of the compound of formula (I) or a pharmaceutically acceptable salt thereof, and a therapeutically effective amount of the compound of formula (II) or a pharmaceutically acceptable salt thereof are administered as a therapeutic combination. The therapeutically effective amount of the compound of formula (I) or a pharmaceutically acceptable salt thereof, and the therapeutically effective amount of the compound of formula (II) or a pharmaceutically acceptable salt thereof may be suitably altered according to symptoms, age, body weight, relative state of health, presence of other drugs, administration method and the like. For example, the typical effective amount for a patient (warm-blooded animal and particularly a human) as the compound of formula (I) or a pharmaceutically acceptable salt thereof, in the case of an oral preparation is preferably 0.1 to 1000 mg, and more preferably 1 to 20 mg per day. In the case of parenteral administration, the typical effective amount of the same is preferably 0.1 to 1000 mg and more preferably 1 to 20 mg per day. Further, the typical effective amount for a patient (warm-blooded animal and particularly a human) as the compound of formula (II) or a pharmaceutically acceptable salt thereof in the case of an oral preparation is preferably 0.1 to 1000 mg, and more preferably 10 to 300 mg per day. In the case of parenteral administration, the typical effective amount of the same is preferably 0.1 to 1000 mg and more preferably 10 to 300 mg per day.

In the method of the present invention, the compound of formula (I) or a pharmaceutically acceptable salt thereof, and the compound of formula (II) or a pharmaceutically acceptable salt thereof can be administered concurrently or separately.

The method of the present invention can be applied to a patient with a proliferative disease including solid tumors, hematological malignancies and hyperplasia, particularly to a patient with a proliferative disease including solid tumors, hematological malignancies and hyperplasia in which both RAS and PI3K pathways are concurrently activated; in which PI3K activities are elevated; or in which MEK is activated.

Also falling within the scope of this invention are methods of treating a patient with a proliferative disease including solid tumors, hematological malignancies and hyperplasia with the combination of the in vivo metabolic products of the compound of formula (I) or a pharmaceutically acceptable salt thereof, and the compound of formula (II) or a pharmaceutically acceptable salt thereof. Such products may result for example from the oxidation, reduction, hydrolysis, amidation, deamidation, esterification, deesterification, enzymatic cleavage, and the like, of the administered compound.

### EXAMPLES

In order to illustrate the invention, the following examples are included. However, these examples do not limit the invention and are only meant to suggest a method of practicing the invention.

### Example 1

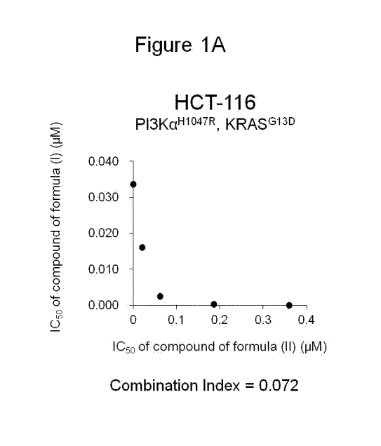

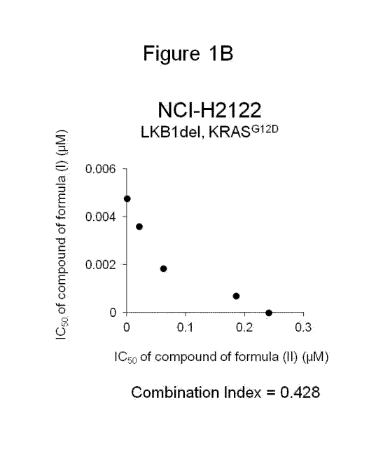

To determine the level of synergy, isobologram analysis was performed and the combination index was calculated by using the compound of formula (I) (MEK inhibitor) and the compound of formula (II) (PI3K inhibitor) according to the method as reported in Pharmacol Rev 58:621-681, 2006. The KRAS and PIK3CA mutated colorectal cancer cell line, HCT116 and the KRAS mutated and LKB1 deleted NSCLC cell line, NCI-H2122 were purchased from ATCC and used in a standard antiproliferative assay detecting with WST-8 (Dojindo). IC₅₀ values of compound of formula (I) and compound of formula (II) were determined and plotted. In both cell lines, the isobolograms and the combination indexes showed that compound of formula (I) synergized with compound of formula (II) (FIG. 1).

### Example 2

To investigate the combination effect on apoptosis induction, compound of formula (I) and compound of formula (II) were tested on Caspase-Glo® 3/7 Assay (Promega).

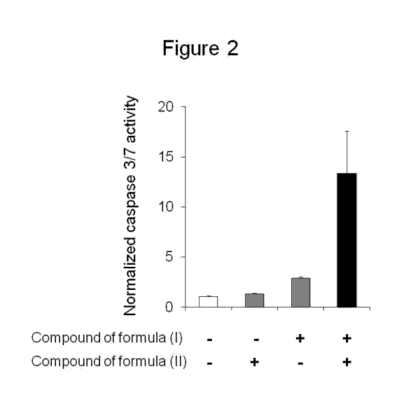

2×10³ of the HCT116 cells were treated with compound of formula (I) alone, compound of formula (II) alone or the combination thereof at 1 μM for 48 hours in 96-well plate. Caspase3/7 induction was determined by measuring luminescence with Caspase-Glo® 3/7 Assay kit (Promega), and normalized with the number of viable cells determined by CellTiter Glo® Assay (Promega). The normalized induction was plotted in FIG. 2. The combination of compound of formula (I) and compound of formula (II) resulted in a marked increase in apoptosis induction compared to each compound alone.

### Example 3

To investigate the combination effect on PI3K and MAPK pathway, compound of formula (I) and compound of formula (II) were tested on HCT116 cells.

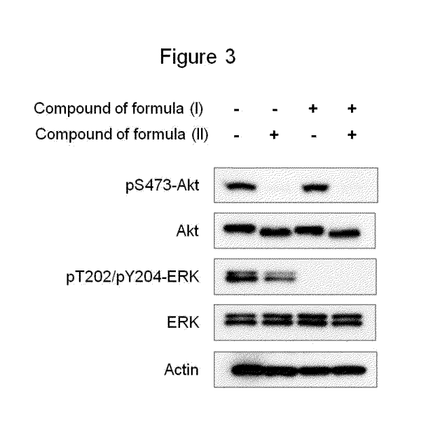

5×10⁵ of cells were treated with compound of formula (I) alone, compound of formula (II) alone or the combination thereof at 1 μM for 24 hours in 6-well plate. After that, HCT116 cells were lysed with Cell Lysis Buffer (Cell Signaling Technology) containing protease and phosphatase inhibitors. The lysates were denatured and then subjected to SDS-PAGE. After electroblotting, the polyvinylidene fluoride membranes were blocked with Blocking One (Nacalai Tesque) for 1 h. Membranes were then incubated with primary antibodies purchased from Cell Signaling Technology Inc., and anti-actin antibody purchased from Santa Cruz Biotechnology over night at 4° C., followed by washing three times with 0.1% Tween 20 (Nacalai Tesque) in TBS buffer (TBS-T buffer; TaKaRa Bio). This was followed by 0.5 to 1 h incubation at room temperature with secondary antibodies (Cell Signaling Technology). Then, membranes were washed four times with TBS-T buffer, followed by incubation with ECL or ECL-plus solutions (GE Healthcare). Signals were detected using LAS-4000 (Fujifilm) according to the manufacturer's instructions. As shown in the FIG. 3, the phosphorylation of respective downstream effectors, ERK and Akt were suppressed. When combined both inhibitors, both effectors were effectively and simultaneously suppressed.

### Example 4

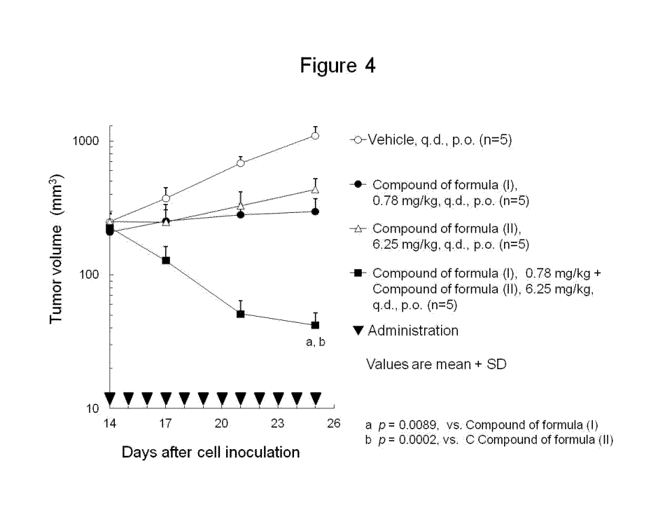

To investigate the combination effect on tumor volume, compound of formula (I) and compound of formula (II) were tested on HCT116 cells. 5×10⁶ cells of the HCT116 cells (PI3Kα^{H1047R}, KRAS^{G13D}) were inoculated subcutaneously into the right flank of each BALB-nu/nu mouse. Tumors were allowed to reach palpable after implantation before initiation of treatment. Vehicle (open circles), compound of formula (I) (0.78 mg/kg, qd., p.o.; filled circles), compound of formula (II) (6.25 mg/kg, qd., p.o.; open triangles), or the combination of compound of formula (I) (0.78 mg/kg, qd., p.o.) and compound of formula (II) (6.25 mg/kg, qd., p.o.) (filled squares) was orally administered once daily for 12 days, from day 14 to day 25. Tumor size was measured by using a gauge twice per week and tumor volume (TV) was calculated using the following formula: TV=ab2/2, where a is the length of the tumor and b is the width. Significant enhancement of antitumor efficacy were observed for the combination of compound of formula (I) and compound of formula (II) compared to compound of formula (I) alone (Tukey's test, p=0.0089) and compound of formula (II) alone (Tukey's test, p=0.0002) at the end of study on day 25 (FIG. 4).

### Example 5

To investigate the combination effect on body weight, the compound of formula (I) and the compound of formula (II) were tested on HCT116 cells.

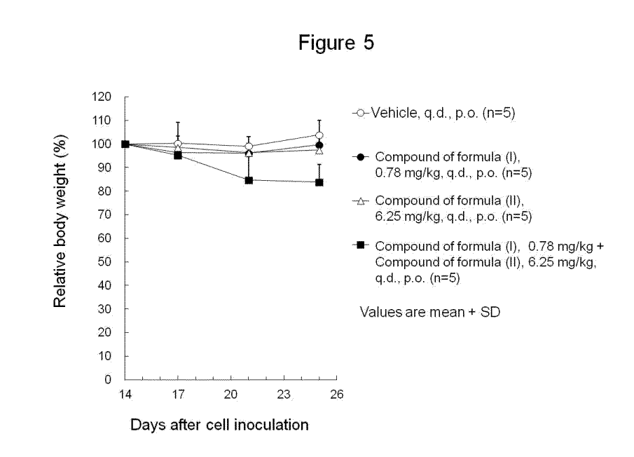

The HCT116 cells (PI3Kα^{H1047R}, KRAS^{G13D}) were inoculated to BALB-nu/nu mice, and vehicle (open circles), compound of formula (I) (filled circles), compound of formula (II) (open triangles), or the combination of compound of formula (I) and compound of formula (II) (filled squares) was orally administered in the same manner as in Example 4. Body weight was measured twice per week and the results are shown in FIG. 5. The combination of compound of formula (I) and compound of formula (II) had no severe adverse effects on body weight (FIG. 5).

1. A method for the treatment of a patient with a proliferative disease including solid tumors, hematological malignancies and hyperplasia comprising administering a therapeutic combination to said patient wherein the therapeutic combination comprises a therapeutically effective amount of a compound of formula (I) or a pharmaceutically acceptable salt thereof, and a therapeutically effective amount of a compound of formula (II) or a pharmaceutically acceptable salt thereof.

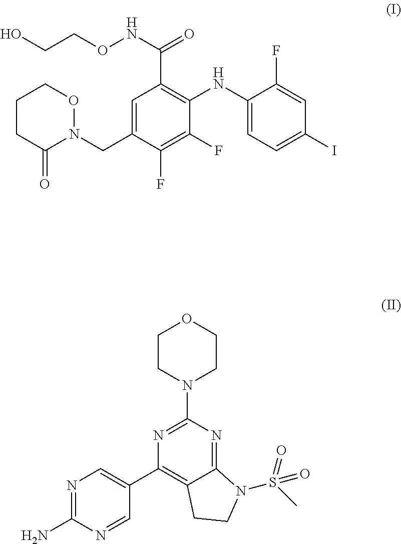 [SMILES: O=C(NOCCO)c1cc(CN2OCCCC2=O)c(F)c(F)c1Nc1ccc(I)cc1F ; CS(=O)(=O)N1CCc2c(-c3cnc(N)nc3)nc(N3CCOCC3)nc21]

2. The method according to claim 1, wherein both RAS and PI3K pathways are concurrently activated in said proliferative disease including solid tumors, hematological malignancies and hyperplasia.

3. The method according to claim 1, wherein PI3K activities are elevated in said proliferative disease including solid tumors, hematological malignancies and hyperplasia.

4. The method according to claim 1, wherein MEK is activated in said proliferative disease including solid tumors, hematological malignancies and hyperplasia.

5. The method according to claim 1, wherein the compound of formula (I) or a pharmaceutically acceptable salt thereof, and the compound of formula (II) or a pharmaceutically acceptable salt thereof are concurrently administered.

6. The method according to claim 1, wherein said patient is orally administered concurrently 0.1 to 1000 mg per day of compound of formula (I) or a pharmaceutically acceptable salt thereof, and 0.1 to 1000 mg per day of compound of formula (II) or a pharmaceutically acceptable salt thereof.

7. A pharmaceutical kit of parts comprising:
a) a pharmaceutical composition comprising a therapeutically effective amount of a compound of formula (I), 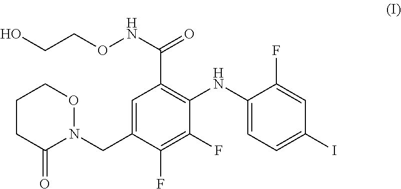 [SMILES: O=C(NOCCO)c1cc(CN2OCCCC2=O)c(F)c(F)c1Nc1ccc(I)cc1F]
or a pharmaceutically acceptable salt thereof,
b) a pharmaceutical composition comprising a therapeutically effective amount of a compound of formula (II), 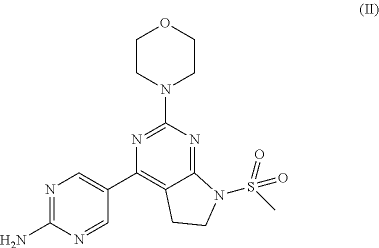 [SMILES: CS(=O)(=O)N1CCc2c(-c3cnc(N)nc3)nc(N3CCOCC3)nc21]
or a pharmaceutically acceptable salt thereof, and optionally
c) instructions for dosing regimen.

8. A method for the treatment of a patient with a proliferative disease including solid tumors, hematological malignancies and hyperplasia comprising the steps of providing the pharmaceutical kit of parts according to claim 7, and administering to said patient a pharmaceutical composition comprising a therapeutically effective amount of the compound of formula (I) or a pharmaceutically acceptable salt thereof, and a pharmaceutical composition comprising a therapeutically effective amount of the compound of formula (II) or a pharmaceutically acceptable salt thereof.
